(19) United States
(12) Patent Application Publication
(10) Pub. No.: US 2014/0121619 A1
(43) Pub. Date: May 1, 2014

(54) EFFECTIVE ODOR CONTROL WITH COATINGS OF DESIGNED POROUS MOLECULES

(71) Applicant: DOW GLOBAL TECHNOLOGIES LLC, Midland, MI (US)

(72) Inventors: Evren Aslan-Guerel, Zuerich (CH); Gert J. Claasen, Richterswil (CH); Rudolf J. Koopmans, Ensiedeln (CH); Scott T. Matteucci, Midland, MI (US)

(73) Assignee: Dow Global Technologies LLC, Midland, MI (US)

(21) Appl. No.: 13/660,297

(22) Filed: Oct. 25, 2012

(51) Int. Cl. A61L 15/46; A61L 15/18

(52) U.S. Cl. CPC A61L 15/46; A61L 15/18
USPC 604/361; 427/2.31; 521/154

(57) ABSTRACT

The present invention relates to coatings for articles comprising a non-zeolitic silica mesoporous structure. The coatings of the present invention have shown an ability to effectively reduce odors.

### FIELD OF THE INVENTION

The present invention relates to coatings for articles comprising a non-zeolitic silica mesoporous structure. The coatings of the present invention have shown an ability to effectively reduce odors.

### BACKGROUND AND SUMMARY OF THE INVENTION

Odor is one of the common complaints in hygiene products. Our dislike of bad smells is the part of the human self-defense mechanism which has been developed across civilizations. For many personal care absorbent articles, medical absorbent articles, and the like, it is desirable to reduce, prevent, or eliminate odors during use. For diapers and other incontinence products, it is desirable to reduce or eliminate the odor of ammonia which is present in urine. For feminine hygiene products, it is desirable to reduce or eliminate the odors of trimethylamine and triethylamine. Other common odor-producing substances include isovaleric acid, dimethyl disulfide, and dimethyl trisulfide.

While the sensing level of the human nose is less than some animal species, it can still detect certain compounds at very low concentration in the atmosphere. For example, trimethylamine can be detected at 0.2 parts per billion by volume. The different level of human nose sensitivity to certain chemicals makes the subject of odor measurements, prevention and reduction difficult. While in general, the mentioned odors in this application are not toxic, they are a nuisance and may invoke an aversion reaction.

Disposable articles are made from thermoplastic polymers in the form of extruded films, foams and nonwovens. An issue with these articles is that they are designed for short term use but may not be disposed of immediately so that there is an opportunity for microorganisms to grow prior to disposal, creating issues with formation of toxins, irritants or odor. Discreetness is a desirable characteristic of an absorbent article to an adult, and part of that discreteness includes the elimination of malodors from the used article.

For hygiene articles, the unpleasant body odors sought to be avoided are mainly organic molecules which have different structures and functional groups, such as amines, acids, alcohols, aldehydes, ketones, phenolics, polycyclics, indoles, aromatics, polyaromatics etc. They can also be made up of sulfur containing functional groups, such as thiol, mercaptan, sulfide and/or disulfide groups.

Typical methods to control odors currently used in the art generally include the use of odor control agents. Odor control agents include odor inhibitors, odor absorbers, odor adsorbers and other compounds which suppress odors. Odor inhibitors prevent the odor from forming. For example, the use of an aminopolycarboxylic acid compound is known to inhibit the formation of ammonia from urea in urine. Odor absorbers and adsorbers remove odor after it is formed. Examples of odor control agents that remove odor by absorption or adsorption include activated carbon, silica, and cyclodextrin.

Acidic odor control agents based on carboxylic acids and their derivatives are used to neutralize or inhibit formation of odors from ammonia and other basic odor-forming compounds Ammonia, released from aqueous ammonium hydroxide, is one of the primary odor-producing substances in urine. One of the drawbacks of acidic odor control agents is they are not inherently durable such that they pass into solution after one or more insults with aqueous liquid, and may acidify the liquid. If some of the acidified aqueous liquid leaks from the absorbent article and passes to the wearer's skin, the wearer may experience itching, rash, and/or other uncomfortable effects.

Previously, acidic odor control agents have been applied to absorbent articles in the form of powders, coatings, and the like, which also tend be easily dissolved. There is a need or desire for absorbent articles having durable odor control agents, which target a broad range of malodorous compounds and which retain their odor control functions and do not pass into solution after one or more insults with aqueous liquid.

In one aspect of the present invention, a coating for an article comprising a non-zeolitic silica mesoporous structure is provided. Mesoporous is defined by IUPAC to have pore sizes between 2 and 50 nm in diameter. The application of porous silica nanoparticles on the surface of nonwovens or other article can advantageously be applied via spray-coating. The porous silica nanoparticles can absorb, neutralize and encapsulate the fecal or other odors on contact. These articles deliver not only improved odor control and fluid handling properties but are able to maintain these properties even upon prolonged wearing time, typically upon ageing of bodily fluid in the article.

### BRIEF DESCRIPTION OF THE DRAWINGS

FIG. 1 shows an image from a scanning electron microscope of MCF particles.

FIG. 2 shows an image from a scanning electron microscope of MCF particles at a closer magnification than FIG. 2.

FIG. 3 shows an image from a scanning electron microscope of MCF particles at a closer magnification than FIG. 3.

FIG. 4 is a graph showing the size distribution for porous silica particles used in the Examples.

### DETAILED DESCRIPTION OF THE INVENTION

### Definitions

The term “mesoporous”, means that the material has pores of diameter within the range of 2 to 50 nm, more preferably 2 to 40 nm, or even 5 to 30 nm. In this respect, pore size is considered as the maximum perpendicular cross-sectional dimension of the pore which can be determined using N₂ adsoption as is generally known in the art, such as described in the article “Recommendations for the Characterization of Porous Solids”, J. Rouquerol, D. Avnir, W. Fairbridge, D. H. Everett, J. H. Haynes, N. Pemicone, J. D. F. Ramsay, K. S. W. Sing, and K. K. Unger; Pure & Appl. Chem.; Vol 66, No. 8; PP 1739-1758; 1994.

For purposes of this invention, the term “non-zeolitic” means material which does not have a peak in x-ray defraction above 6 degrees. In one embodiment of the invention the mesoporous silica structure does not have any peak in x-ray diffraction above 1 degree.

Test Methods

Sensory Measurement of Odors

It is the nature of very odorous compounds that at low concentration they are detected by the human nose even when less odorous gases are present at higher concentrations. The high sensitivity and discrimination makes the nose for its purpose far superior to any chemical or instrumental method yet developed. Hence teams of human observers—as sensory instruments—are required still for work on the detection and assessment of odors, particularly because odor nuisance itself is determined by the sense of smell. However, whilst this section describes sensory techniques for quantitative and reproducible measurements of odors by means of panelists and olfactometers, the following section then describes instrumental techniques which can be used to separate and identify the constituents of an odor, something the nose cannot always do.

For an odorant the perceived intensity of odor is proportional to a fractional power of the concentration, and a range of standard concentrations of a selected odorant in air could be used to provide a scale of intensities for direct comparison with samples of odorous gases collected at their sources. But there are limits to practicable or tolerable concentrations and to the volumes of samples; also, adaption occurs rapidly at high concentrations and a comparative technique involves duplication of test equipment and effort.

The basic principle of olfactometry is that a sample of odorous gas is diluted with odor free air to various extents in order to the number of dilutions required for odor to be just perceived by 50% of the members of a panel.

Methods for odorous analysis are thus normally based in gas chromatographic separation following by physical detection and measurement of the separated components. In the gas chromatographic process the sample is blended into an inert carrier gas stream and flows through a column containing a material with properties chosen so that different components are retarded to different degrees.

The effectiveness of the deodorizing release liner of the present invention may be measured with the headspace gas chromatography test. The effectiveness of the ink in removing odors may also be measured in terms of “Relative adsorption efficiency”, which determined using headspace gas chromatography and measured in terms of milligrams of odor adsorbed per gram of the ink.

Headspace Determination of Odor Control

The effectiveness of the odor control can be demonstrated by determining the percent reduction in the concentration of common odorant molecules in the headspace surrounding the sample (non-woven).

Headspace Gas Chromatography/Mass Spectrometry (HS-GC/MS) analysis can be used to measure changes in the presence and concentration of molecules in the headspace.

Approximately 0.2 g of nonwoven (coated or uncoated) sample is weighed into a 20 mL Agilent headspace vial, using an analytical balance. The vial is closed with a magnetic crimp cap, containing silicone coated septa. One mL of a gas standard solution, containing known amounts of pyridine and diethylsulfide as described below, are added to the vial. The vials are equilibrated for 15 minutes at 40° C. using a Gerstel MPS-2 headspace sampler. After equilibration 1.0 mL of gas is injected into the GC/MS system.

A control sample (blank) containing no odor control material is analyzed along with samples modified with odor control material.

GC/MS Headspace testing is conducted using an Agilent 7890A Gas Chromatograph equipped with an Agilent 5975C mass spectrometer. A Varian VF-1701, 30 m×0.32 mm, column with a 0.5 μm film thickness is used for the separation of the components. A standard SGE focus liner is used in the split/split less inlet. Temperatures for the mass spectrometer are set to 280° C. for the transfer line, 150° C. for the quadrupole and 230° C. for the source. The mass spectrometer is configured for EI mode, scanning from 10-550 m/z.

GC oven temperature programming is held at 50° C. for 2 minutes, and then heated at a rate of 10° C./min to 80° C. Total Ion Chromatograms (TIC) for the mass range 10-550 are collected after a 1 minute solvent delay. The integrated peak area of the response of odor molecules is obtained from the TIC and is used in determining the percent reduction of the odor molecules as compared to a control sample with no odor control material present (blank).

### Gas Standard Preparation:

A 1 Liter Tedlar bag is filled with helium. To this bag, 10 μL of pyridine and diethylsulfide are added. The calculated concentrations are shown below.

TABLE 1
Odorant concentrations in standard

| | Concentration |
|---|---|
| Component | (ppm, v/v) |
| Diethylsulfide | 2301 |
| Pyridine | 3079 |

Coating

The coatings for use in the present invention have a non-zeolitic silica mesoporous structure. The mesoporous structures have a porosity in the range of from 2-50 nm, more preferably 2 to 40 nm, or even 5 to 30 nm. Preferably the coating has a surface area of at least 200 m²/g, preferably at least 300 m²/g, and even more preferably at least 400 m²/g as determined by physisorption isotherm data using the Brunauer-Emmett-Teller (BET) method which is readily known in the art (see also the Rouquerol article mentioned above). It should be understood that during the process to apply the particles as a coating, or during shipping or use of the article, the surface and/or structure of the particles may be affected such that the surface area of the coating at a given time, may be different from the surface area of the particles prior to application as a coating.

One particularly preferred form of the coating is a mesoporous cellular foam (or “MCF”). Such MCFs can be synthesized by oil-in water micro-emulsion templating approach, allowing for precise control of MCF final properties. Typically, the MCF particles are synthesized as taught in U.S. Pat. No. 6,641,657 U.S. Pat. No. 6,506,485, U.S. Pat. No. 6,592,764, US20100048390, or Schmidt-Winkel et al, J. Am. Chem. Soc. 121, 254-255 (1999).

The MCFs or other suitable non-zeolitic silica mesoporous structures, can advantageously be added to the surface of an article by first forming a dispersion of the particles in a solvent, then applying the solvent to the article and then removing the solvent, leaving the particles as a coating. Alternatively, a composition comprising a mixture of a water-soluble or water-dispersible binder material and a water-insoluble MCF or other suitable non-zeolitic silica mesoporous structure as odor controlling agent can be used. The preferred binder materials can be selected from the group of materials consisting of hydroxymethyl celluloses, hydroxyethyl celluloses, hydroxylpropyl celluloses, alkyl substituted celluloses, dextrin derivatives and mixtures thereof.

Other particles which may also be used to form coatings include silicas and aluminosilicates that are mesoporous structures having ordered pores but an amorphous host, for instance such as those taught in U.S. Pat. No. 6,592,764, U.S. Pat. No. 5,238,676, or U.S. Pat. No. 5,266,541.

Article

The article which can be coated for use in the present invention can be a finished article or a component of such article such as a fiber, film, foam, absorbent core, or a nonwoven fabric. These articles can be made of many different materials, including polyolefins.

One type of article commonly used in hygiene articles which could be aided by the present invention are fibers. Such fibers include polymeric materials as well as cellulosic fibers, and may be monocomponent fibers or bicomponent fibers as is generally known in the art.

Another particularly preferred article contemplated for use in the present invention is a nonwoven fabric which is comprised of fibers. Such fabrics are preferable made from polyolefin fiber, whether monocomponent or bicomponent. Any nonwoven structure in the art can be used with the present invention. Such structures may include those formed by a variety of processes, such as, for example, air laying processes, meltblowing processes, spunbonding processes and carding processes, including bonded carded web processes.

As used herein, the term “meltblown”, refers to the process of extruding a molten thermoplastic material through a plurality of fine, usually circular, die capillaries as molten threads or filaments into a high velocity gas (e.g., air) stream which attenuates the filaments of molten thermoplastic material to reduce their diameter, which may be to a microfiber diameter. Thereafter, the meltblown fibers are carried by the high velocity gas stream and are deposited on a collecting surface to form a web of randomly dispersed meltblown fibers.

As used herein, the term “spunbonded”, refers to the process of extruding a molten thermoplastic material as filaments from a plurality of fine, usually circular, capillaries of a spinneret with the diameter of the extruded filaments then being rapidly reduced by drawing the fibers and collecting the fibers on a substrate. The nonwoven web may comprise a single web, such as a spunbond web, a carded web, an airlaid web, a spunlaced web, or a meltblown web. However, because of the relative strengths and weaknesses associated with the different processes and materials used to make nonwoven fabrics, composite structures of more than one layer are often used in order to achieve a better balance of properties. Such structures are often identified by letters designating the various lays such as SM for a two layer structure consisting of a spunbond layer and a meltblown layer, SMS for a three layer structure, or more generically SXₙS structures, where X can be independently a spunbond layer, a carded layer, an airlaid layer, a spunlaced layer, or a meltblown layer and n can be any number. In order to maintain structural integrity of such composite structures, the layers must be bonded together. Common methods of bonding include point bonding, adhesive lamination, and other methods known to those skilled in the art.

It is also contemplated that breathable and/or non-breathable films, including extrusion coated films, may be used as layers in a multilayered structure with the nonwoven web. All of these structures may be used in the present invention.

It is contemplated that coated fibers of the present invention may be used to make nonwoven fabrics and/or structures, which may then further be coated according to the present invention.

The coatings of the present can be applied to the article in any way known to the art. One such method, preferred for some applications involves spray drying a dispersion of the particles onto the surface of the article. MCF as odor controlling agent may be distributed together or separately, homogeneously or nonhomogeneously, over the entire absorbent article or at least one layer of the topsheet or in at least one layer of the backsheet, or in at least one layer of the core or any mixture thereof. MCF may be distributed homogeneously or non homogeneously on the whole surface of the desired layer or layers, or on one or several area of the surface layer/layers to which it is positioned (e.g. central area and/or surrounding area like the edges of a layer of the absorbent article) or mixtures thereof. The coating may advantageously be present on the article at between 0.05 g/m² to 3 g/m², preferably between 0.30 g/m² and 2.0 g/m², and even more preferably between 0.50 g/m² and 1.5 g/m², where g/m² indicates grams of coating and m² is defined as meters squared of the article prior to coating.

The coated articles of the present invention can be characterized by their ability to remove gasses in the Headspace Determination. For example it is preferred that the resulting article exhibits and improvement of at least 50%, more preferably at least 90%, in removal of ammonia gas in the headspace as compared to a similar article without the coating.

### EXAMPLES

Mesoporous cellular foam (MCF) particles are synthesized as follows: a micro emulsion sample is first made by dissolving 10 g of PEO-PPO-PEO tri-block copolymer (Pluronic P123 from BASF, EO20-PO70-EO20) in 375 ml of 1.6 M HCl at room temperature. 15 g of 1,3,5-trimethylbenzene is slowly added to the tri-block copolymer solution, and the mixture is kept at 40° C. for 1 hour. Next, 22 g of tetraethyl othosilicate is added to the mixture. After approximately 24 hours at 40° C., the milky mixture is transferred to an autoclave and aged at 100° C. for another 24 hours to produce MCF particles, which were subsequently filtered out and washed with deionized water. After drying at room temperature for 24 hours, the surfactant is removed from the resulting particles by calcinations at 550° C. for 8 hours in air flow.

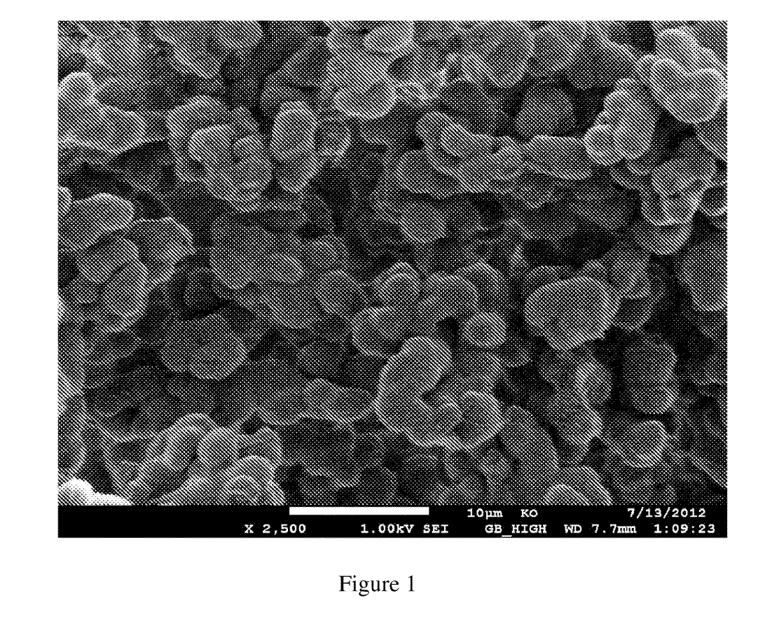

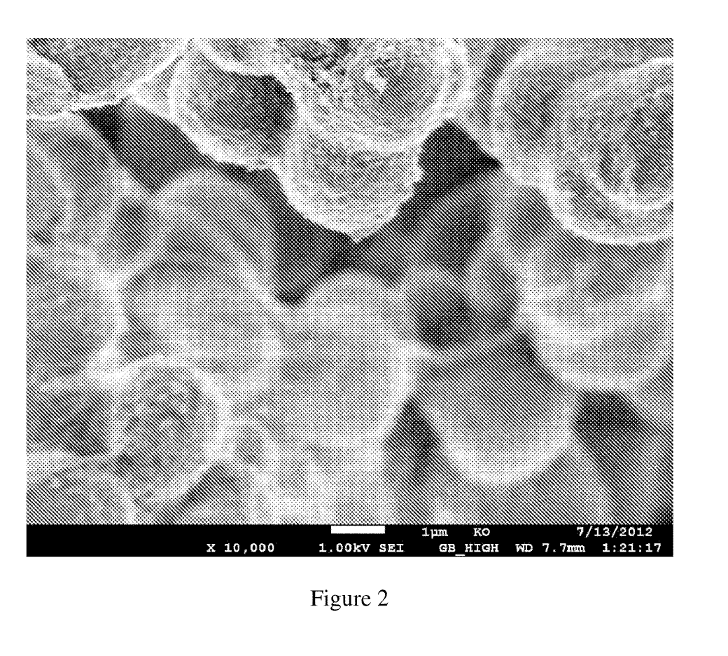

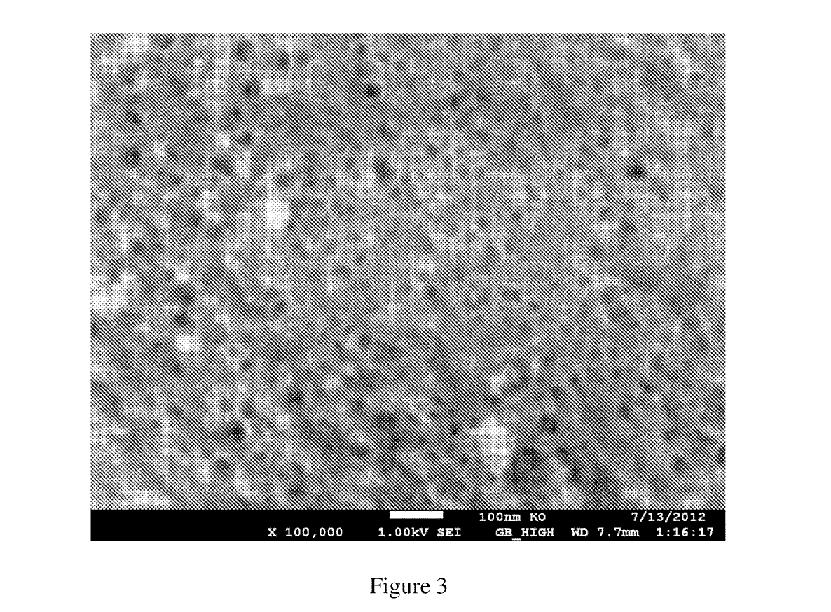

The highly porous silica particle used in this work is a type of mesoporous cellular foam, which represent a new class of aerogel-like, three dimensional, continuous, ultra-large pore mesoporous materials that are synthesized with well controlled and uniformly sized pores. FIGS. 1-3 show images from a scanning electron microscope at different magnifications. FIG. 2 indicates that the particles are about 1 to 2 microns in size. FIG. 3, the highest magnification surface micrograph taken for the MCF particle, shows that the pores on the particle range between 10 to 35 nm.

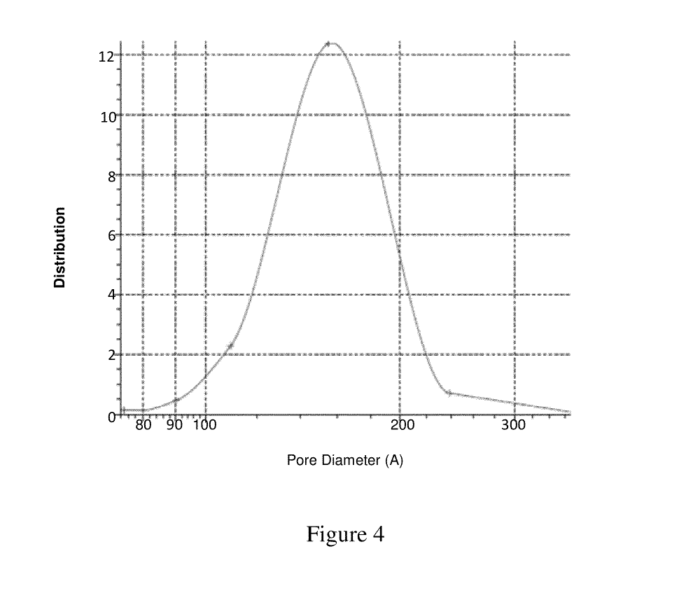

Additionally, nitrogen adsorption isotherm of the porous silica particles was determined and used to characterize their pore size distribution. The BET absorption surface area was estimated to be 650 m²/g from the adsorption isotherm. Furthermore, as shown in FIG. 4, pores in these silica particles range from 80 to 300 Å and center at 180 Å.

### Example 1

### MCF on Polyethylene Spunbond Nonwoven

An aqueous dispersion of the MCF particles is prepared using a high shear mixer. Typical shear time 30 minutes. The dispersion is sprayed on a polyethylene spunbond nonwoven (20 gsm). Fibers used to produced the nonwoven have a dpf (denier/filament) of 1.4 den.

The dispersion is then transferred into a adjustable spray bottle. The surface of polyethylene spunbond nonwoven is modified with MCF dispersion at a level of about 0.8 g/m². The modified nonwoven is dried at room temperature (24° C.) for 12 h. The samples are then tested with head space methodology described above.

Table 2 contains the data measured for modified NWs. In the table, the sample designation Ref refer to the control experiment (that is, an uncoated nonwoven having a basis weight of 20 gsm) and the designation with M refer to the modified nonwoven as described above. The results shown are the amount of the indicated odorant remaining (for example if the odorant was added at a 100 ppm level, and after exposure to the sample the amount of odorant in the headspace was found to be 75 ppm, a value of 75% would be reported).

TABLE 2
Odorant concentration in the head space

| | | |
|---|---|---|
| | Diethylsulfide | Pyridine |
| | concentration | concentration |
| Sample | (% remaining) | (% remaining) |

PE - 20 gsm

| | | |
|---|---|---|
| Ref | 98 | 100 |
| M | 56 | 0 |

### Example 2

### MCF on Polyethylene Spunbond Nonwoven

An aqueous dispersion of MCF particles is prepared using a high shear mixer. Typical shear time 30 minutes. The dispersion is sprayed on a polyethylene spunbond nonwoven (80 gsm). Fibers used to produced the nonwoven have a dpf (denier/filament) of 1.4 den.

The dispersion is then transferred into a adjustable spray bottle. The surface of polyethylene spunbond nonwoven is modified with MCF dispersion (at a level of about 0.8 g/m² MCF). The modified nonwoven is dried at room temperature (24° C.) for 12 h. The samples are then tested with head space methodology described above.

Table 3 contains the data measured for modified NWs. In the table, the sample designation Ref refer to a control experiment, of the same nonwoven except at a basis weight of 20 gsm, without any coating applied. The designation with M refers to the modification described above. The results shown are the amount of the indicated odorant remaining (for example if the odorant was added at a 100 ppm level, and after exposure to the sample the amount of odorant in the headspace was found to be 75 ppm, a value of 75% would be reported).

TABLE 3
Odorant concentration in the head space

| | | |
|---|---|---|
| | Diethylsulfide | Pyridine |
| | concentration | concentration |
| Sample | (% remaining) | (% remaining) |

PE - 80 gsm

| | | |
|---|---|---|
| Ref | 98 | 100 |
| M | 59 | 1 |

### Example 3

### MCF on a Bicomponent Spunbond Nonwoven

An aqueous dispersion of MCF particles is prepared using a high shear mixer. Typical shear time 30 minutes. The dispersion is sprayed on a polyethylene/polypropylene bico (50/50 wt %) spunbond nonwoven having a basis weight of 20 gsm. Fibers used to produced the nonwoven have a dpf (denier/filament) of 1.4 den.

The dispersion is then transferred into a adjustable spray bottle. The surface of bicomponent spunbond nonwoven is modified with MCF dispersion (about 0.8 g/m²). The modified nonwoven is dried at room temperature (24° C.) for 12 h. The samples are then tested with head space methodology described above.

Table 4 contains the data measured for modified NWs. In the table, the sample designation Ref refer to the control experiment and the designation with M refer to the modification described above. The results shown are the amount of the indicated odorant remaining (for example if the odorant was added at a 100 ppm level, and after exposure to the sample the amount of odorant in the headspace was found to be 75 ppm, a value of 75% would be reported).

TABLE 4
Odorant concentration in the head space

| | | |
|---|---|---|
| | Diethylsulfide | Pyridine |
| | concentration | concentration |
| Sample | (%) | (%) |

PE/PP - 20 gsm

| | | |
|---|---|---|
| Ref | 98 | 101 |
| M | 55 | 1 |

### Example 4

### MCF Combined Mixture on Bicomponent Spunbond Nonwovens

An aqueous dispersion of MCF particles and active carbon are prepared using a high shear mixer. Typical shear time 30 minutes. The dispersion is sprayed on a bicomponent spunbond nonwoven (polyethylene/polypropylene bico (50/50 wt %)). Fibers used to produced the nonwoven have a dpf (denier/filament) of 1.4 den.

The dispersion is then transferred into a adjustable spray bottle. The surface of polyethylene spunbond nonwoven is modified with MCF and active carbon dispersions about 0.8 g/m² and about 0.8 g/m², respectively. The modified nonwoven is dried at room temperature (24° C.) for 12 h. The samples are then tested with head space methodology described above.

Table 5 contains the data measured for modified NWs. In the table, the sample designation Ref refer to the control experiment and the designation with M refer to the modification described above. The results shown are the amount of the indicated odorant remaining (for example if the odorant was added at a 100 ppm level, and after exposure to the sample the amount of odorant in the headspace was found to be 75 ppm, a value of 75% would be reported).

TABLE 5
Odorant concentration in the head space

| | | |
|---|---|---|
| | Diethylsulfide | Pyridine |
| | concentration | concentration |
| Sample | (%) | (%) |

PE/PP - 20 gsm

| | | |
|---|---|---|
| Ref | 98 | 101 |
| M | 37 | 1 |

### Example 5

### MCF on Hydrophilic Treated Bicomponent Spunbond Nonwoven

An aqueous dispersion of MCF particles is prepared using a high shear mixer. Typical shear time is 30 minutes. The dispersion is sprayed on a hydrophilic modified bicomponent spunbond nonwoven (polyethylene/polypropylene bico (50/50 wt %). Fibers used to produce the nonwoven have a dpf (denier/filament) of 1.4 den.

The dispersion is then transferred into an adjustable spray bottle. The surface of hydrophilic bicomponent spunbond nonwoven is modified with MCF dispersion (about 0.8 g/m²). The modified nonwoven is dried at room temperature (24° C.) for 12 h. The samples are then tested with head space methodology described above.

Table 6 contains the data measured for modified NWs. In the table, the sample designation Ref refer to the control experiment (using the untreated polyethylene spunbond nonwoven) and the designation with M refer to the modification described for Example 5 above. The results shown are the amount of the indicated odorant remaining (for example if the odorant was added at a 100 ppm level, and after exposure to the sample the amount of odorant in the headspace was found to be 75 ppm, a value of 75% would be reported).

TABLE 6
Odorant concentration in the head space

| | | |
|---|---|---|
| | Diethylsulfide | Pyridine |
| | concentration | concentration |
| Sample | (%) | (%) |

PP/PE bico

| | | |
|---|---|---|
| Ref | 98 | 100 |
| Hydrophilic | 77 | 74 |
| treated-ref | | |
| Hydrophilic | 55 | 1 |
| treated-M | | |

1. A coating for an article, the coating comprising an odor control agent consisting essentially of a non-zeolitic silica mesoporous structure.

2. The coating of claim 1 wherein the article is a polyolefin based nonwoven.

3. The coating of claim 1 wherein the article is a fiber.

4. The coating of claim 3 wherein the fiber is a polyolefin based fiber.

5. The coating of claim 3 wherein the fiber is a cellulosic fiber.

6. The coating of claim 3 wherein the fiber is part of a nonwoven layer.

7. The coating of claim 1 wherein the article is an absorbent core

8. The coating of claim 1 wherein the article is an absorbent core and the absorbent core is a cellulosic substrate of a fibrous nature.

9. The coating of claim 1 wherein the resulting article exhibits an improvement of at least 50% in removal of ammonia gas in the headspace as compared to a similar article without the coating.

10. The coating of claim 9 wherein the improvement is at least 90%

11. A method of improving the odor control of a hygiene article comprising the step of coating the hygiene article with an odor control agent consisting essentially of a non-zeolitic silica mesoporous structure.

12. The method of claim 11 wherein the article exhibits a reduction of at least 50% of ammonia gas in the headspace

13. An article suitable for removing one or more target gases, wherein the article comprises a surface and the surface has a coating comprising an odor control agent consisting essentially of a non-zeolitic silica mesoporous structure.

14. The coating of claim 1 wherein the mesoporous structure is a cellular foam.

15. The coating of claim 1 wherein the mesoporous structure has ordered pores but an amorphous host.

16. The coating of claim 1 wherein the coating comprises non-zeolitic mesoporous silica structure having a precoating surface area of at least 200 m²/g.

17. The article of claim 13, wherein the coated article comprises between 0.05 to 3 g of coating per meter squared of uncoated article.
